(19) United States
(12) Patent Application Publication
(10) Pub. No.: US 2006/0076184 A1
(43) Pub. Date: Apr. 13, 2006

(54) Alleviating stethoscopically-caused contamination

(76) Inventor: Jeffrey L. Robinson, Salt Lake City, UT (US)

Correspondence Address: Lynn G. Foster, 602 East 300 South, Salt Lake City, UT, 84102, US

(21) Appl. No.: 10/961,802

(22) Filed: Oct. 8, 2004

(51) Int. Cl. A61B 7/02

(52) U.S. Cl. 181/131

(57) ABSTRACT

Disposable sterile coverings impervious to bacteria and viruses, and related methods, are disclosed, the coverings temporarily insulating the operative base surface of the diaphragm of a traditional high quality, repeat use stethoscope. Each covering is discarded after a single use, thereby preventing cross bacterial and viral infections, including those caused by highly resistant bacteria, between patients on which the same stethoscope is successively used.

### FIELD OF INVENTION

The present invention relates generally to cross contamination caused by use of the same medical instrument on different medical patients and more particularly to alleviation of cross bacteria and viral infections between patients heretofore caused by successive use of the same stethoscope, using a disposable protective cover, which is impervious to bacteria and viruses.

### BACKGROUND

There is a rising incidence of highly resistant bacteria in hospital settings. The common procedure to prevent spread of these bacteria is the use of isolation precautions, including use of gowns, gloves, and disposable “non-critical” items. These non-critical items include disposable stethoscopes. The current disposable stethoscopes of choice are inexpensive, of low quality and produce imprecise patient data, making it very difficult to adequately assess patient maladies, thus impairing the quality of care. No other solution, which defeats spread of bacteria while attempting to accommodate the stethoscope needs of a medical patient is currently available, with one exception, i.e. use of standard non-disposable stethoscopes where expensive diaphragms, which are coated with an antibiotic, are discarded and replaced with new ones between patients.

### BRIEF SUMMARY AND OBJECTS OF THE INVENTION

In brief summary, the present invention overcomes or substantially alleviates problems of cross infections caused by repeated use of the same stethoscope on two or more medical patients, while obtaining high quality patient data. Disposable sterile coverings, and related methods, are provided for temporarily insulating the operative surface of the base of the diaphragmof a traditional high quality, repeat use stethoscope from contact with the patient, such that the covering is discarded after a single use and replaced by a new disposable covering, thereby alleviating cross bacterial and viral infections, including those caused by highly resistant bacteria, between patients on which the same stethoscope is successively used.

With the foregoing in mind, it is a primary object to overcome or substantially alleviate problems of cross infections caused by use of the same stethoscope on successive patients.

Another object of value is the provision of a disposable covering, and related methods, for temporarily insulating the operative surface of the base of the diaphragm of a repeat use stethoscope from direct contact with the patient, such that the covering is discarded after a single use and replaced by a new disposable covering to alleviate cross bacterial and/or viral infections between patients on which the same stethoscope is successively used.

These and other objects and features of the present invention will be apparent from the detailed description taken with reference to the accompanying drawings.

### BRIEF DESCRIPTION OF THE DRAWINGS

FIG. 1 is a flow diagram of one way the present invention is implemented to protect a repeat use stethoscope from cross contamination between patients, using disposable covers;

FIGS. 2-5 are additional disposable cover embodiments, in accordance with the present invention, for protecting a diaphragm of a repeat use stethoscope;

FIG. 6 is still another disposable cover embodiment for temporarily encapsulating both the head and the diaphragm of a repeat use stethoscope; and

FIGS. 7-10 are additional cover embodiments, in accordance with the present invention, where the cover both removable and adhered to the operative base surface of the diaphragm of a repeat use stethoscope.

### DETAILED DESCRIPTION OF THE ILLUSTRATED EMBODIMENTS

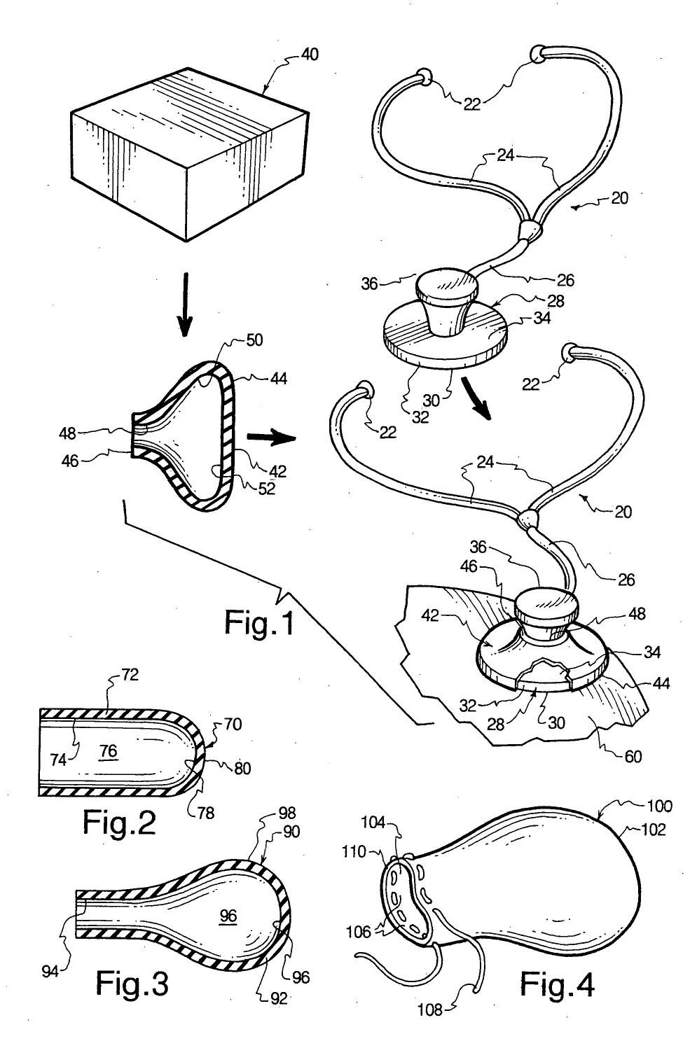

Reference is now made to the drawings in detail where like numerals are used to designate like parts throughout. The invention involves alleviating cross bacterial and viral infections between patients upon whom a repeat use stethoscope is used. Such a stethoscope is illustrated in FIG. 1, generally designated 20. Stethoscope 20 comprises standard ear receptors 22 a V-shaped neck piece 24, a stem 26, a diaphragm 28 comprising a layer 32 including an operative base surface 30. The diaphragm 28 comprises a top surface 34, which is conventionally joined to a bell head 36.

The present invention comprises disposable coverings, impervious to bacteria and viruses which insulate diaphragm surface 28 from direct contact with the patient. In some embodiments, the cover engages both surfaces 30 and 34 of the diaphragm 28. In other embodiments, the covering comprises a pad adhered primarily to the surface 30.

It is contemplated that a supply of covering in accordance with the present invention would be made available in containers, such as container 40, shown in FIG. 1. With continued reference to FIG. 1, a covering 42 is shown, in accordance with the present invention which has been removed from container 40. Preferably, cover 42 is comprised of latex material so that it is stretchable to accommodate placement over the diaphragm 28 and comprises memory which constricts the proximal end of covering 42 to retain the covering 42 over the diaphragm.

Covering 42 is illustrated in FIG. 1 as being bulbous in its configuration so as to comprise an enlarged distal end 44 and a narrow proximal end 46 defining a small unstressed proximal opening 48. Opening 48 is substantially smaller than the diameteral size of the diaphragm 28 but covering 42 accommodates stretching from the position shown in FIG. 1 adjacent to the container 40 around the diaphragm 28 coming to rest when released in the installed position over the diaphragm 28 also shown in conjunction with stethoscope 20 in FIG. 1. The covering 42 is illustrated as comprising a wall of uniform thickness defining a hollow interior 50 and is shaped so as to comprise an interior, generally flat surface 52 which contiguously engages diaphragm surface 30, when installed on the stethoscope in the manner shown in FIG. 1.

Any other suitable cover configuration which is single use may be used to insulate the diaphragm surface 30 from the patient 60 in order to alleviate cross contamination between patients on which the same stethoscope is successively used. For example, the cylindrically shaped cover, generally designated 70, in FIG. 2 may be used. Cover 70 is illustrated as comprising a balloon like wall 72 of uniform thickness defining a proximal opening 74 and an interior blind bore 76 terminating at distal interior surface 78. The cover 70 is placed over the diaphragm 28 in essentially the same manner as described above by stretching the wall 72 and opening 74 and inserting the diaphragm 28 through the stretched opening 74 until diaphragm surface 30 is contiguous with covering surface 78. At that point, distal exterior surface 80 is flat and contiguous with diaphragm surface 30. Surface 80 may be placed upon the patient 60 (FIG. 1) to obtain stethoscopic data in the usual way. Covering 70 may be comprised of latex or other similar material which is stretchable, has memory and is impervious to bacteria and viruses.

Another suitable covering, generally designated 90, is illustrated in FIG. 3. Cover 90 is bulbous shaped comprising a round wall 92 of uniform thickness although uniform thickness, is not mandatory, the wall 92 terminating in a small diameter distal opening 94 providing access to the hollow interior 96 of the cover 90 defined by the wall 92. Wall 92 comprises an exterior surface 98. The covering 90 is installed upon the diaphragm 28 of the stethoscope by stretching in the manner explained above and functions in fundamentally the same way as covers 42 and 70, described above. In all cases, after a single use, each covering 42, 70 and 90 will be removed from the diaphragm 28 using manual force and discarded carrying with it any bacterial or viral contamination received from the patient 60 during use.

Reference is now made to FIG. 4 which illustrates a further cover embodiment in accordance with the principles of the present invention, generally designated 100. Cover 100 is illustrated as being generally beaker shaped and may or may not comprise elastomeric material capable of a substantial stretch. Cover 100 comprises an exposed flat distal surface 102, a hollow interior 104, a proximal opening 106 and a draw string 108 fitted through proximal apertures 110 by which the covering 100 may be temporarily but removably secured in position around the diaphragm 28 primarily by tying the ends of drawstring 108 together in a conventional manner. The covering 100 receives the diaphragm 28 and is used as described above.

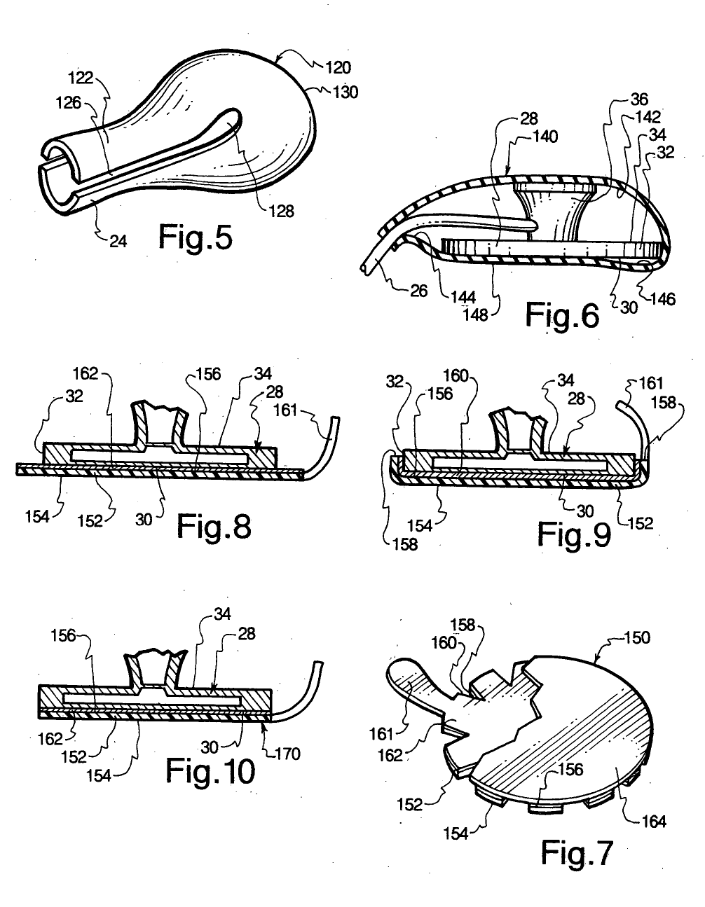

Reference is made to FIG. 5 which illustrates a covering, generally designated 120, embodying principles in accordance with the present invention and comprising elongated handles 122 and 124, which are separated by a slot 126, the handles accommodating placement of the cover 120 over and removal of the cover from the diaphragm 28. When operative, the diaphragm comes to rest at the hollow interior 128 with the surface 30 being contiguous with the interior surface at the distal end of the cover 120 such that exterior surface 130 is placed contiguous with the patient 60 to obtain stethoscopic data in the usual way. The material from which cover 120 is made can be a highly stretchable elastomer or less stretchable and largely of fixed dimensions.

Reference is made to FIG. 6 which illustrates a further cover, generally designated 140, in accordance with the principles of the present invention. Cover 140 is illustrated as, when installed, encapsulating both the diaphragm 28 and the head 36 of the stethoscope 20 and comprises a bulbous though eccentric shape comprising a hollow interior 142, an access opening 144 into the hollow interior 142, an interior surface 146 shown as being contiguous with surface 30 of the diaphragm 28 and an exterior surface 148 for direct contact with the patient during use of the stethoscope. Covering 140 is manually removed by stretching and discarding after a single use.

Reference is now made to FIG. 7, which illustrates a further embodiment, generally designated as 150, in accordance with the present invention by which the stethoscopic surface 30 is protected against contact with bacteria and viruses, so as to alleviate cross contamination between successive patients. The cover 150, stored in a sterile package, comprises a yieldable disk 152, which is illustrated as being of uniform thickness and comprises an exposed surface 154 and a protected surface 156. The edge 158 of the disk 152 is illustrated as having spaced V-shaped notches 160. A layer of suitable releasable adhesive 160 is superimposed upon surface 156.

Disk 152 is illustrated as having a side tab 161 for purposes yet to be explained. At the time of manufacture, the adhesive 162 superimposed upon surface 156 is covered by a suitable protective layer 164 comprised of Kraft paper or other suitable material.

The cover 150 is sized and shaped so as to be diametrically somewhat larger than the diameter of the diaphragm 28. In use, the protective layer 64 is manually removed and the adhesive layer 162 is positioned directly adjacent to surface 30 of the diaphragm 28 in more or less concentric relationship and the two are brought contiguously together so as to achieve the position illustrated in FIG. 8.

Next, the peripherary of the layer 152 is folded essentially through 90 degrees into contact with diaphragm edge surface 32 from the position of FIG. 8 to the position of FIG. 9. The notches 160 accommodate the indicated rotation such that the peripheral edge is adhesively secured

1. A combination:
a repeat use stethoscope comprising a diaphragm having an operative base surface;
a disposable cover insulating the operative base surface from a patient during stethoscopic use;
such that the disposable cover becomes contiguous with a medical patient during stethoscope use so that bacterial and/or viral contamination, if any, occurs on and is discarded with the cover prior to later use of the stethoscope on another patient.

2. A combination according to claim 1 wherein the disposable cover temporarily encloses the diaphragm.

3. A combination according to claim 1 wherein the cover is formed of elastomeric material, is stretched over the diaphragm and is held in position by a compression force due to the memory of the elastomeric material.

4. A combination according to claim 1 wherein the elastomeric material comprises latex.

5. A combination according to claim 1 wherein the stethoscope comprises a head and the cover is sized and shaped so that it substantially encloses the head and the diaphragm.

6. A combination according to claim 2 further comprising a fastener by which at least in part the cover is held in position temporarily over the diaphragm in contiguous relation.

7. A combination according to claim 6 wherein the fastener comprises at least one draw string.

8. A combination according to claim 2 wherein at least one handle for manually manipulating the cover onto and off from the diaphragm.

9. A combination according to claim 2 wherein the cover comprises a stress free shape selected from the group consisting of cylindrical, bulbous and dog bone.

10. A combination according to claim 1 wherein the cover comprises a pad removably adhered to the operative base surface of the diaphragm.

11. A combination according to claim 10 wherein the pad comprises a tab for removing the cover from the operative base surface after a single use.

12. A disposable cover for placement at the distal end of a repeat use stethoscope in superimposed relation over an operative base surface of a diaphragm of the stethoscope, the cover comprising a patient engaging surface, a diaphragm engaging surface and an element by which the cover is temporarily but removably secured to the diaphragm.

13. A disposable cover according to claim 12 wherein the patient engaging surface of the cover comprises part of a layer having a second surface by which the cover is removably adhered to the operative base surface of the diaphragm.

14. A disposable cover according to claim 12 wherein the cover comprises a material imperious to bacteria and viruses.

15. A disposable cover according to claim 14 wherein the material is selected from the group comprising a stretchable elastomer with memory and fixed dimension pad adapted to adhere to the base surface.

16. A method of alleviating cross bacteria and/or viral infections caused by multiple uses of a repeat use stethoscope comprising the acts of:
(a) superimposing a cover impervious to bacteria and viruses over an operative base surface of a diaphragm of the stethoscope in but retained removable relationship;
(b) placing the cover contiguous with a medical patient;
(c) gaining patient data through use of the stethoscope;
(d) removing the cover from the patient; and
(e) removing the cover from the stethoscope and discarding the cover.

17. A method according to claim 16 wherein the cover is elstomeric and step (a) comprises wrapping the elastomeric cover substantially over the diaphragm so that the cover is held in the superimposed position by the memory of the elastomeric cover.

18. A method according to claim 16 wherein step (a) comprises removably adhering the cover to the operative base surface.

19. A method according to claim 16 wherein step (a) comprises removable adhering the cover to the operative base surface using an additional releasable fastener.
